(19) United States
(12) Patent Application Publication
(10) Pub. No.: US 2018/0055453 A1
(43) Pub. Date: Mar. 1, 2018

(54) METHOD OF ESTIMATING RESPIRATORY RATE AND ELECTRONIC APPARATUS THEREOF

(71) Applicant: HTC Corporation, Taoyuan City (TW)

(72) Inventors: Po-Lei Lee, Taoyuan City (TW); De-Zhang Peng, Taoyuan City (TW); Huan-Hsin Li, Taoyuan City (TW)

(73) Assignee: HTC Corporation, Taoyuan City (TW)

(21) Appl. No.: 15/247,842

(22) Filed: Aug. 25, 2016

(51) Int. Cl. A61B 5/00; A61B 5/0205; A61B 5/0402

(52) U.S. Cl. CPC A61B 5/7278; A61B 5/725; A61B 5/02416; A61B 5/0402; A61B 5/7253; A61B 5/0205

(57) ABSTRACT

A method of estimating a respiratory rate and an electronic apparatus are provided. The method includes following steps. A physiological signal is obtained. A wave signal associated with baseline drift is extracted from the physiological signal. A wave number of the wave signal is counted, and the respiratory rate is estimated according to the wave number.

### BACKGROUND

### Technical Field

The present disclosure relates to an estimation method and an electronic apparatus thereof, and more particularly relates to a method of estimating respiratory rate and an electronic apparatus thereof.

### Description of Related Art

As medical technology improves every day, countless lives have been saved and the overall quality of life continues to improve over time. Especially, development of medical devices and equipment has made significant contribution to the improvement of the medical technology. Nowadays, many wearable devices and monitor systems have been applied in continuously measuring vital signs of a person. Through the measured vital signs, physical health condition of the person could be assessed.

Respiratory rate is one of the vital signs widely used as an early and sensitive indicator of deterioration and serious illness. A common technique applied in the respiratory rate measurement involves measuring transthoracic impedance through electrodes. However, in the previously mentioned technique, the electrodes are necessary, and wearing the electrodes brings inconvenience to the person during the respiratory rate measurement. Other techniques, such as the Electrocardiography Derived (ECG-Derived) Respiration and Photoplethysmography Derived (PPG-Derived) Respiration, have been proposed for measuring the respiratory rate, but those techniques rely upon calculating peaks of the ECG signals and the PPG signals, which leads to heavy computation. Further, for the person having lower heart rate, ECG-Derived Respiration and PPG-Derived Respiration cannot determine the respiratory rate precisely. Therefore, how to provide a convenient and precise method for estimating the respiratory rate without heavy computation is an issue for relevant researchers to work on.

### SUMMARY

The present disclosure provides a method of estimating respiratory rate and an electronic apparatus thereof, in which the baseline drift of a physiological signal is utilized for estimating the respiratory rate.

An embodiment of the disclosure provides a method of estimating a respiratory rate. The method includes following steps. A physiological signal is obtained. A wave signal associated with baseline drift is extracted from the physiological signal. A wave number of the wave signal is counted. The respiratory rate is estimated according to the wave number.

Another embodiment of the disclosure provides an electronic apparatus adapted to estimate a respiratory rate. The electronic apparatus includes a memory and a processor. A plurality of modules are stored in the memory. The processor coupled to the memory obtains a physiological signal and executes the modules loaded from the memory. The loaded modules include an extraction module, a count module and an estimation module. The extraction module extracts a wave signal associated with baseline drift from the physiological signal. The count module counts a wave number of the wave signal, and the estimation module estimates the respiratory rate according to the wave number.

Another embodiment of the disclosure provides a method of estimating a respiratory rate. The method includes following steps. A physiological signal is obtained. A wave signal associated with baseline drift is extracted from the physiological signal. A time-frequency transform is performed on the wave signal to obtain a frequency spectrum. The respiratory rate is estimated according to the frequency spectrum, wherein a frequency value having a maximal amplitude represents the respiratory rate.

Based on the above, in the method of estimating the respiratory rate of an examinee and the electronic apparatus provided in the present disclosure, the wave signal associated with baseline drift is extracted from the physiological signal of the examinee, and the wave number of the wave signal is counted for estimating the respiratory rate of the examinee. To be more specific, the wave number represents the drift condition of the physiological signal in the time domain, and the respiratory rate is derived based on the drift condition of the physiological signal. As such, the respiratory rate of the examinee is precisely estimated without heavy computation. Another method of estimating the respiratory rate of the examinee using time-frequency transform technique is also provided.

To make the above features and advantages of the present disclosure more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a block diagram of an electronic apparatus according to an embodiment of the present disclosure.

FIG. 2 is a flowchart illustrating a method of estimating a respiratory rate of an examinee according to an embodiment of the present disclosure.

FIG. 3 is a schematic diagram of counting a wave number of a wave signal according to an embodiment of the present disclosure.

FIG. 4 is a flowchart illustrating another method of estimating a respiratory rate of an examinee according to an embodiment of the present disclosure.

### DESCRIPTION OF THE EMBODIMENTS

Reference will now be made in detail to the present preferred embodiments of the disclosure, examples of which are illustrated in the accompanying drawings. Wherever possible, the same reference numbers are used in the drawings and the description to refer to the same or like parts.

The baseline drift is a common phenomenon that could be normally seen in physiological signals measured from an examinee, especially in an electrocardiography (ECG) signal and a photoplethysmography (PPG) signal. In addition, baseline drift is usually caused by low frequency interference happened during the measurement of the physiological signals, such as human breathing. Usually, the baseline drift is regarded as noise in the physiological signals, so it is mostly eliminated or reduced from the physiological signals. However, in the present application, taking advantage of the baseline drift, a respiratory rate of an examinee may be estimated precisely without heavy computation.

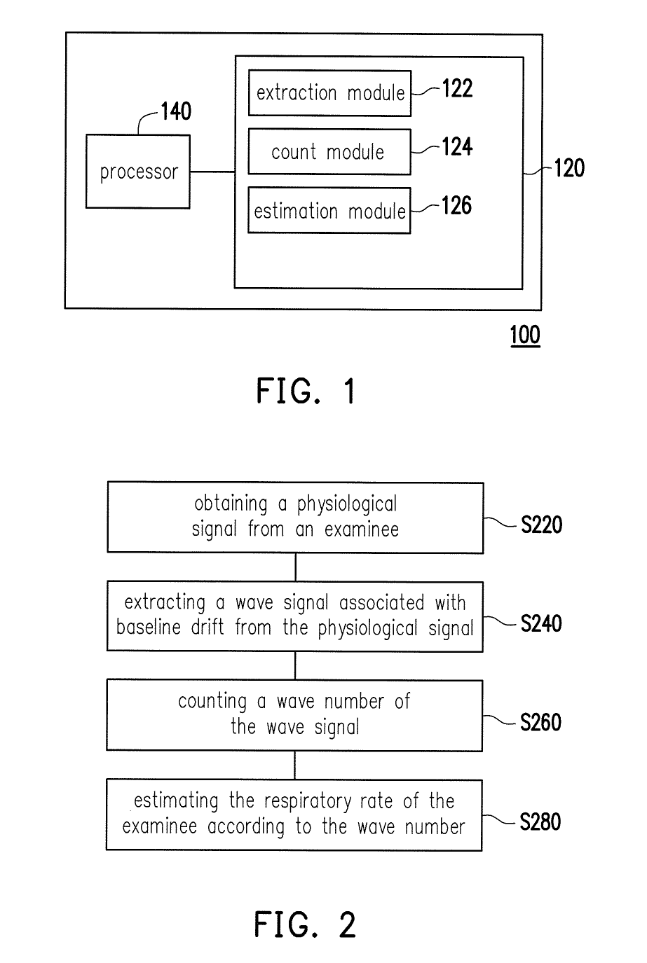

FIG. 1 is a block diagram of an electronic apparatus according to an embodiment of the present disclosure. In the present embodiment, the method of estimating the respiratory rate of the examinee is adapted to the electronic device 100 illustrated in FIG. 1, but the apparatus adaptable for the method is not limited herein.

Referring to FIG. 1, the electronic apparatus 100 includes a memory 120 and a processor 140. The electronic apparatus 100 may be a personal computer, a laptop, a tablet computer, a smart mobile device or a wearable electronic apparatus implemented in a form of a sticker, a wristband, a heart rate band, a helmet, a necklace, a watch, a ring, a bracelet, a clothes, or a belt, etc., capable of being worn by an examinee, but it is not limited thereto.

In an embodiment of the present disclosure, the memory 120 is configured to store data, modules, applications or programs, and accessible by the processor 140. The memory 120 may be, for example, a hard disk drive (HDD), a volatile memory, a non-volatile memory or a combination thereof. The modules stored in the memory 120 include an extraction module 122, a count module 124 and an estimation module 126. The processor 140 is capable of loading and executing the afore-mentioned modules, and operation details thereof will be described later in following embodiments.

In an embodiment of the present disclosure, the processor 140 coupled to the memory 120 may include a central processing unit (CPU), a programmable general purpose or special purpose microprocessor, an application specific integrated circuit (ASIC), a field programmable array (FPGA), a programmable logic device (PLD), or other similar devices or a combination thereof. The processor 140 is capable of accessing the memory 120 and executing the modules loaded from the memory 120.

Moreover, in another embodiment of the present disclosure, the electronic apparatus 100 may further include at least one sensor, a display device, a transmission interface or a combination thereof, which are not illustrated in FIG. 1. Specifically, the at least one sensor may be utilized for detecting the physiological signals by the electronic apparatus 100, the display device may be utilized for displaying information and data obtained by the electronic apparatus 100, and the transmission interface may be utilized for communicating with other apparatuses by the electronic apparatus 100.

In an embodiment of the present disclosure, the sensor may include an electrocardiogram (ECG/EKG), a PPG sensor, a blood pressure meter, or any other physiological data measuring sensor, or a combination of the above devices, but it is not limited thereto. The display device may be, for example, a liquid crystal display (LCD) device, a light-emitting diode (LED) display device, an organic light-emitting diode (OLED) display device, a plasma display device or other types of display devices. Further, the transmission interface supports various wireless communication standards and wire communication standards such as Bluetooth protocol, Wireless Fidelity (Wi-Fi) protocol, WiMAX (Worldwide Interoperability for Microwave Access) protocol, Zigbee protocol, LTE (Long Term Evolution) protocol, Asymmetric Digital Subscriber Line (ADSL) communication standard.

FIG. 2 is a flowchart illustrating a method of estimating a respiratory rate of an examinee according to an embodiment of the present disclosure. Referring to FIG. 2, the method of the present embodiment is adapted to the electronic apparatus 100 shown in FIG. 1, but it is not limited herein. The method of the present embodiment is described below with reference of various components and modules of the electronic apparatus 100.

In the present embodiment, a physiological signal from the examinee within a time interval is first obtained by processor 140 (step S220). The physiological signal may be an ECG signal, a PPG signal or a combination thereof. In an embodiment of the present disclosure, the processor 140 obtains the physiological signal from other sensing devices such as an ECG device and a PPG device through the transmission interface. However, in another embodiment of the present disclosure, the processor 140 may directly obtain the physiological signal from the at least one sensor built in the electronic apparatus 100.

After the physiological signal is obtained, the extraction module 122 loaded and executed by the processor 140 extracts a wave signal associated with baseline drift from the physiological signal (step S240). To be more specific, since the baseline drift of the physiological signal may be caused by the human breathing, the extraction module 122 extracts the wave signal associated with baseline drift for estimating the respiratory rate of the examinee.

The wave signal could be extracted from the physiological signal by means of the filtering, the wavelet transform or the empirical mode decomposition (EMD), but it is not limited herein. To be more specific, in an embodiment, for extracting the wave signal from the physiological signal, the extraction module 122 filters the physiological signal by passing the physiological signal through at least one band-pass filter to obtain the wave signal, wherein a passband of the band-pass filter comprises a range from approximately 0.1 (Hz) to 0.6 (Hz), but it is not limited herein. Furthermore, in an embodiment of the present disclosure, before extracting the wave signal from the physiological signal, the extraction module 122 further down-samples the physiological signal. The physiological signal is down-sampled at a sampling frequency, which is approximately 20 Hz, but it is not limited herein. The sampling frequency should be larger than an upper limit of a breath frequency range which is for human beings.

From above, it is considered that the wave signal is extracted from a sub-band of a frequency band of the physiological signal. Referring to FIG. 2, after the wave signal is extracted, the count module 124 loaded and executed by the processor 140 counts a wave number of the wave signal (step S260). Specifically, the wave signal is associated with the baseline drift which caused by the breathing of the examinee. Thus, the wave number of the wave signal represents to the number of the times that the examinee breathes within the time interval.

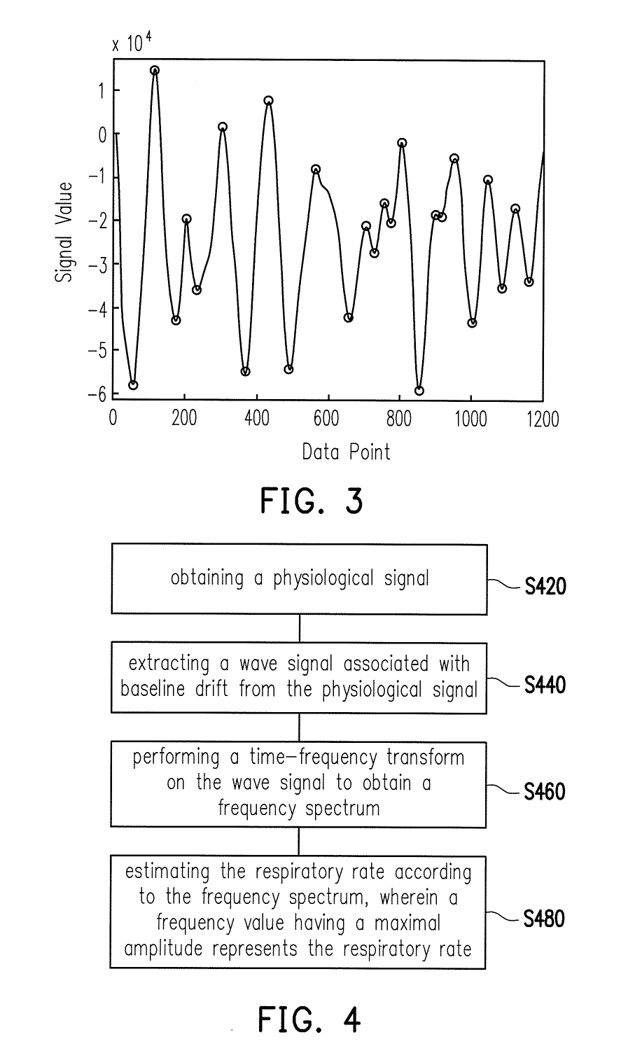

FIG. 3 is a schematic diagram of counting a wave number of a wave signal according to an embodiment of the present disclosure. In an embodiment of the present disclosure, the wave number of the wave signal is counted by the count module 124 by finding the number of local maximums in the wave signal, the number of local minimums in the wave signal or the number of pairs of the local maximum and the local minimum among the data points in the wave signal. As shown in FIG. 3, the count module 124 counts the wave number of the wave signal by means of the peak-trough detection, but it is not limited herein. In an embodiment of the present disclosure, the wave number of the wave signal is counted by the count module 124 by finding a total number of all the local maximums and the local minimums and dividing that total number by 2. After the division, if the calculated wave number has the decimal number, round down the calculated wave number to an integer. In another embodiment of the present disclosure, the wave number of the wave signal is counted by the count module 124 by shifting the wave signal to correspond with zero value and finding the number of zero-crossing points in the wave signal. To be more specific, the count module 124 counts the wave number of the wave signal by means of the zero-crossing detection. It should be noted that, the count module 124 filters the wave signal by passing the wave signal through at least one high pass filter, or adds a mean value of the wave signal to the wave signal, or subtracts the mean value of the wave signal from the wave signal, so as to shift the wave signal to correspond with the zero value.

Referring to FIG. 1 and FIG. 2, after the number of the wave in the wave signal is counted, the estimation module 126 loaded and executed by the processor 140 estimates the respiratory rate of the examinee according to the wave number (step S280). Specifically, since the wave number of the wave signal represents the number of the times that the examinee breathes within the time interval, the respiratory rate of the examinee may be derived from the wave number and the time interval. In an embodiment of the present disclosure, respiratory rate of the examinee may be shown on the display device of the electronic apparatus 100 for the examinee. Further, when the respiratory rate of the examinee is fall into an abnormal range, the electronic apparatus 100 outputs a warning message for the examinee.

It should be noted that, in the embodiments mentioned above, the respiratory rate of the examinee is estimated through the wave number and the time interval defined in the time domain. By the wave number, even minute variation of the breath condition of the examinee could be aware, so the respiratory rate of the examinee could be precisely estimated based on the wave number.

FIG. 4 is a flowchart illustrating another method of estimating a respiratory rate of an examinee according to an embodiment of the present disclosure. The method shown in FIG. 4 is also adapted to an electronic apparatus, such as the electronic apparatus 100 shown in FIG. 1. Referring to FIG. 4, in the present embodiment, instead of estimating the respiratory rate based on the wave number of the wave number counted in the time domain, the processor may 120 may load and execute a module for performing a time-frequency transform on the wave signal to obtain a frequency spectrum (step S460). The time-frequency transform may be Fourier Transform, Fast Fourier Transform (FFT) or other transform method, but it is not limited herein. After the frequency spectrum of the wave signal is obtained, the estimation module 126 estimates the respiratory rate according to the frequency spectrum, wherein a frequency value having a maximal amplitude in the frequency spectrum represents the respiratory rate (step S480). The other steps and the detail of the estimation method shown in FIG. 4 could be referred from the description of the estimation method shown in FIG. 1, so those are not repeated herein.

In summary, in the method of estimating the respiratory rate of the examinee and the electronic apparatus provided in the present disclosure, the wave signal associated with baseline drift is extracted from the physiological signal of the examinee, and the wave number of the wave signal is counted for estimating the respiratory rate of the examinee. To be more specific, the wave number represents the drift condition of the physiological signal in the time domain, and the respiratory rate is derived based on the drift condition of the physiological signal. As such, the respiratory rate of the examinee is precisely estimated without heavy computation. Another method of estimating the respiratory rate of the examinee using time-frequency transform technique is also provided.

Although the disclosure has been described with reference to the above embodiments, it will be apparent to one of ordinary skill in the art that modifications to the described embodiments may be made without departing from the spirit of the disclosure. Accordingly, the scope of the disclosure will be defined by the attached claims and not by the above detailed descriptions.

What is claimed is:

1. A method of estimating a respiratory rate, comprising:
obtaining a physiological signal;
extracting a wave signal associated with baseline drift from the physiological signal;
counting a wave number of the wave signal; and
estimating the respiratory rate according to the wave number.

2. The method as claimed in claim 1, wherein the step of extracting the wave signal from the physiological signal comprises:
extracting the wave signal from a sub-band of a frequency band of the physiological signal.

3. The method as claimed in claim 1, wherein the step of extracting the wave signal from the physiological signal comprises:
filtering the physiological signal by passing the physiological signal through at least one band-pass filter to obtain the wave signal, wherein a passband of the band-pass filter comprises a range from approximately 0.1 (Hz) to 0.6 (Hz).

4. The method as claimed in claim 1, wherein before the step of extracting the wave signal from the physiological signal, the method further comprises:
down-sampling the physiological signal before extracting the wave signal from the physiological signal at a sampling frequency, wherein the sampling frequency is approximately 20 (Hz).

5. The method as claimed in claim 1, wherein the wave number of the wave signal is counted by finding number of local maximums in the wave signal, number of local minimums in the wave signal, number of pairs of the local maximum and the local minimum in the wave signal, or half of total number of the local maximums and the local minimums in the wave signal.

6. The method as claimed in claim 1, wherein the step of counting the wave number of the wave signal comprises:
shifting the wave signal to correspond with zero value; and
finding number of zero-crossing points in the wave signal.

7. The method as claimed in claim 6, wherein the step of shifting the wave signal to correspond with the zero value comprises:
filtering the wave signal by passing the wave signal through at least one high pass filter, or adding a mean value of the wave signal to the wave signal, or subtracting the mean value of the wave signal from the wave signal.

8. The method as claimed in claim 1, wherein the physiological signal comprises at least one of an electrocardiography (ECG) signal and a photoplethysmography (PPG) signal.

9. An electronic apparatus, comprising:
a memory, storing a plurality of modules; and
a processor, coupled to the memory, obtaining a physiological signal and executing the modules loaded from the memory, the loaded modules comprises:
an extraction module, extracting a wave signal associated with baseline drift from the physiological signal;
a count module, counting a wave number of the wave signal; and
an estimation module, estimating a respiratory rate according to the wave number.

10. The electronic apparatus as claimed in claim 9, wherein the extraction module extracts the wave signal from a sub-band of a frequency band of the physiological signal.

11. The electronic apparatus as claimed in claim 9, wherein the extraction module filters the physiological signal by passing the physiological signal through at least one band-pass filter to obtain the wave signal, wherein a passband of the band-pass filter comprises a range from approximately 0.1 (Hz) to 0.6 (Hz).

12. The electronic apparatus as claimed in claim 9, wherein the extraction module further down-samples the physiological signal before extracting the wave signal from the physiological signal at a sampling frequency, wherein the sampling frequency is approximately 20 (Hz).

13. The electronic apparatus as claimed in claim 9, wherein the wave number of the wave signal is counted by the count module by finding number of local maximums in the wave signal, number of local minimums in the wave signal, number of pairs of the local maximum and the local minimum in the wave signal, or half of total number of the local maximums and the local minimums in the wave signal.

14. The electronic apparatus as claimed in claim 9, wherein the count module shifts the wave signal to correspond with zero value and finds number of zero-crossing points in the wave signal to count the wave number of the wave signal.

15. The electronic apparatus as claimed in claim 14, wherein the count module filters the wave signal by passing the wave signal through at least one high pass filter, or adds a mean value of the wave signal to the wave signal, or subtracts the mean value of the wave signal from the wave signal, so as to shift the wave signal to correspond with the zero value.

16. The electronic apparatus as claimed in claim 9, wherein the physiological signal comprises at least one of an electrocardiography (ECG) signal and a photoplethysmography (PPG) signal.

17. A method of estimating a respiratory rate, comprising:
obtaining a physiological signal;
extracting a wave signal associated with baseline drift from the physiological signal;
performing a time-frequency transform on the wave signal to obtain a frequency spectrum; and
estimating the respiratory rate according to the frequency spectrum, wherein a frequency value having a maximal amplitude represents the respiratory rate.

18. The method as claimed in claim 17, wherein the step of extracting the wave signal from the physiological signal comprises:
extracting the wave signal from a sub-band of a frequency band of the physiological signal.

19. The method as claimed in claim 17, wherein the step of extracting the wave signal from the physiological signal comprises:
filtering the physiological signal by passing the physiological signal through at least one band-pass filter to obtain the wave signal, wherein a passband of the band-pass filter comprises a range from approximately 0.1 (Hz) to 0.6 (Hz).

20. The method as claimed in claim 17, wherein before the step of extracting the wave signal from the physiological signal, the method further comprises:
down-sampling the physiological signal before extracting the wave signal from the physiological signal at a sampling frequency, wherein the sampling frequency is approximately 20 (Hz).
